(19) United States
(12) Patent Application Publication
(10) Pub. No.: US 2017/0348252 A1
(43) Pub. Date: Dec. 7, 2017

(54) Preparation and Use of Ketamine Derivatives and Ketamine Modifiers in the Treatment of Pain

(71) Applicant: Robert KENT, Orlando, FL (US)

(72) Inventor: Robert KENT, Orlando, FL (US)

(21) Appl. No.: 15/494,386

(22) Filed: Apr. 21, 2017

Related U.S. Application Data
(60) Provisional application No. 62/325,574, filed on Apr. 21, 2016.

(51) Int. Cl. A61K 31/14; A61K 31/135; A61K 47/51

(52) U.S. Cl. CPC A61K 31/14; A61K 47/51; A61K 31/135

(57) ABSTRACT

Disclosed herein are ketamine derivatives that have been modified to reduce or prevent transference across the blood brain barrier. The ketamine derivatives will allow the reduction of pain in the patients without complications associated with psychotropic effects typically associated with unmodified ketamine.

### BACKGROUND

There are several known methods for treating and managing pain. There are also varying types of pain. Pain may be peripheral or central, or a combination of both, with peripheral pain being the most difficult to treat.

Neuropathic pain accounts for much of the peripheral pain experienced by patients. The International Association for the Study of Pain defines neuropathic pain as pain which is initiated or caused by a primary lesion or dysfunction in the nervous system due to disordered peripheral or central nerves. The disorder can be caused by compression, transection, infiltration, ischemia, or metabolic injury to neuronal cell bodies, or in combination. Because peripheral neuropathic pain is difficult to treat, existing methods of treatment focus on occupational therapy or helping patients cope psychologically.

In general, medications for the treatment of pain operate on different receptors and are prescribed in accordance with the type of pain a patient experiences. For instance, pain may be managed using anti-inflammatories which treat inflammation via specific cytokine channels at the injury. Opioids on the other hand bind to mu receptors, but work centrally only.

Gabapentin® and Lyrica® are neuromodulators which have been used to treat neuropathic pain, including peripheral neuropathy. Unfortunately, they are less effective at treating peripheral neuropathy because they are GABA channel/receptor medications which work centrally at the level of the spinal cord, but to a lesser degree in the periphery.

One avenue that is not targeted by current neuropathic pain medications are those which operate via the N-methyl-D-aspartate (NMDA) receptor Ca²⁺ channel pore (the “NMDA receptor” or “NMDA”). NMDA is a glutamate channel which appears to be the primary mechanism of Ketamine (Anirudda Pai et al., Contin. Educ. Anaesth. Crit. Care Pain (2007) 7(2): 59-63). Ketamine (2-(2-chlorophenyl)-2-methylamino cyclohexanone) is a general anesthetic usually administered intramuscularly or intravenously for induction of anesthesia. Ketamine is also known to have analgesic properties. (Domino et al., 1965, Clin. Pharmacol. Ther. 6:279). Mechanisms for the blockade of the NMDA receptor by Ketamine is described in Orser, B., et al., Multiple Mechanisms of Ketamine Blockade of N-methyl-D-aspartate Receptors, Anesthesiology 1997; 86(4):903-917.doi:

It was originally believed humans only had central NMDA receptors. Recent research reveals NMDA receptors exist peripherally. Because of the existence of peripheral NMDA receptors, Ketamine can be a treatment to affect one of the main pain receptors in neuropathic pain at the site, most notably peripheral neuropathic pain. This has been attempted by using low doses of Ketamine to treat Complex Regional Pain Syndrome (CPRS). See, Correll, G. et al., Pain Med. (2004) 5(3): 263-275. The Correll study resulted in some patients experiencing complete relief, although the duration of the relief and side effects experienced by patients varied widely. Other research indicates that intravenously administered Ketamine showed lower evidence for effectiveness for the treatment of CPRS. O'Connell N. et al., Interventions for treating pain and disability in adults with complex regional pain syndrome: an overview of systematic reviews, Cochrane Database of Systematic Reviews 2013, (2013) (4): Art. No.: CD009416. DOI: 10.1002/14651858.CD009416.pub2. While wide scale studies are not available, there have been multiple studies of topical Ketamine as a possible treatment for multiple different neuropathies. There is a study currently being designed by Moffitt Cancer Center in Tampa, Fla. to identify the benefit of topical ketamine to treat chemotherapy induced peripheral neuropathy, a common cause of pain and dysfunction after receiving chemotherapy. This study was undertaken based on both anecdotal results and case studies demonstrating certain favorable results in treating peripheral neuropathies with topical ketamine, further demonstrating the possibility of peripheral NMDA receptors and the potential benefit of peripherally acting NMDA mediated medications for neuropathic processes.

### SUMMARY

It has been realized that one of the reasons for the lack of effectiveness of using Ketamine to treat peripheral pain—or at least its limited use—relates to the psychotropic side effects of Ketamine, because in both the Correll and O'Connell studies, patients experienced moderate to severe psychotropic side effects. Further, it has been realized that these side effects are due largely in part by the ability of Ketamine to cross the blood brain barrier and directly affect brain function. Because of these side effects, providers continue to offer GABA medications (such as Gabapentin® and Lyrica®) rather than Ketamine to treat peripheral neuropathy, despite their limitations.

Disclosed herein is the recognition that one of the challenges to the use of Ketamine for the treatment of neuropathic pain as described above is that the pain mitigated is offset by the various psychotropic side effects caused by Ketamine, and in particular the ability of Ketamine to cross the blood brain barrier (the “BBB”). Embodiments of the disclosure include the following:

(1) a composition for treating neuropathic pain, including peripheral neuropathy, with medications whose effects are mediated by NMDA receptors. A particular embodiment is a composition for treating neuropathic pain, including peripheral neuropathy, comprising
(a) a Ketamine derivative and/or homolog modified so as to exhibit overall greater net molecular dipole moment than the unmodified form of Ketamine;
(b) Ketamine directly or indirectly linked with a water soluble carrier;

(2) a process for the synthesis and/or recovery of the composition above.

(3) methods and formulations for treating neuropathic pain, including peripheral neuropathy, with minimal psychotropic side effects comprising administering to a patient Q-N-Ketamine or other quaternary ammonium salts of Ketamine, either singly or in combination with one or more other drugs, using various formulations.

### BRIEF DESCRIPTION OF THE DRAWINGS

FIG. 1 is an reaction scheme for the synthesis of Q-N-Ketamine, in accordance with an embodiment.

FIG. 2 shows a reaction for the formation of Q-N-Ketamine from a tertiary amine derivative of Ketamine, in accordance with an embodiment.

FIG. 3 shows the formation of Q-N-Ketamine in accordance with Example 2 of the Detailed Description, in accordance with an embodiment.

FIG. 4 shows an exemplar reaction scheme for the synthesis of Ketamine.

FIG. 5 shows a reaction for the formation of Q-N-Ketamine, in accordance with an embodiment described in Example 3 of the Detailed Description.

### DETAILED DESCRIPTION

In this disclosure, reference is made to particular features (including method steps) of the invention. It is to be understood that the disclosure of the invention in this specification includes all possible combinations of such particular features. For example, where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other features, ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

This invention may be embodied in many different forms and should not be construed as limited to the embodiments set forth herein. Rather, these embodiments are provided so that this disclosure will be thorough and complete, and will convey the scope of the invention to those skilled in the art.

Embodiments of the invention involve the synthesis of and methods of use of Ketamine homologs for the treatment of pain. A particular embodiment is the preparation of quaternary ammonium salts of Ketamine. For the purpose of this disclosure, and for convenience, these quaternary ammonium salts of Ketamine shall be referred to collectively as “Q-N-Ketamine” Q-N-Ketamine may be prepared by first converting the secondary amine in Ketamine to a tertiary amine, followed by the addition of a hydrocarbon side chain to the resulting tertiary amine to yield a quaternary ammonium salt. Example processes are provided in the Examples, below.

Another particular embodiment is the method of use of a topical formulation of Q-N-Ketamine for the treatment of neuropathic pain.

One advantage to the compositions disclosed is their reduced ability to cross the BBB. Ketamine, in either its original form or in any previously known synthesized homolog, crosses the BBB. Most molecules are simply unable to pass the BBB largely due to of the lack of fenestrae (gaps) between the endothelial cells which make up the capillaries of the brain. (In the body's peripheral capillaries, these gaps between the endothelial cells of the capillary walls are what allow water, ions, and small solutes to move across the membrane.). Passage into the BBB therefore must involve either (1) a molecule's ability to pass through the capillary's endothelial cell walls themselves or (2) a temporary disruption of the BBB capillary endothelial cell walls to create temporary fenestrae. The latter has been achieved by injecting hyperosmotic fluid along with the drug, causing the endothelial cells to temporarily shrink due to water loss, thus creating gaps between the capillary's endothelial cells through which a target molecule may pass (King, A., Breaking through the Blood Brain Barrier, Chem. World (2011) June: 36-39). Achieving passage of a molecule through the actual cell wall of the epithelial cells is described in, for instance, Jain, K., Nanobiotechnology-Based Strategies for Crossing the Blood-Brain Barrier, Nanomedicine. 2012; 7(8):1225-123. The main methods for passing the BBB involve the following:

1. Simple passive diffusion of molecules with the endothelial cell wall of the brain capillaries. Molecules with low molecular mass and a high degree of lipid solubility favor crossing the BBB by simple passive diffusion. This is a non-saturable mechanism that depends on the drug “melding” into the cell membrane on the capillary side and then out again into the brain;

2. Binding of the molecule to specialized receptors on the endothelial cell walls, which permit the entry of the molecule into the cell; and

3. Binding of the molecule to transport proteins which permit crossing of the BBB. For example, glucose and amino acids the brain needs for everyday metabolism are carried across by special transport proteins.

Ketamine has a relatively low molecular mass. It is also highly lipid soluble (5-10 times that of thiopental) (Anirudda Pai et al., Contin. Educ. Anaesth. Crit. Care Pain (2007) 7(2): 59-63) and is thus able to cross the BBB rapidly by diffusion.

For a molecule to have a high degree of lipid solubility, it must be nonpolar. One way to reduce a drug's lipid solubility is to increase the drug's polarity. In order for a drug to be “polar,” the molecule must contain polar bonds where the sum of all the bond's dipole moments is not zero. In other words, in order for a molecule to be polar, it must at minimum have atom(s) that exhibit a partially positive and partially negative charge.

For a molecule to be polar, its three-dimensional representation must be studied. A symmetric molecule, for example CO₂, is nonpolar despite the presence of the highly electronegative atom, Oxygen, because the dipole moments across the molecule “cancel” one another out across the central carbon. Asymmetrical molecules, on the other hand tend to exhibit a net dipole moment and the molecule is therefore polar.

In drug chemistry, the polarity of a substance is measured by its partition coefficient in a two phase system consisting of 1-octanol and water, where Polarity, P, is defined as

P=[amount of drug dissolved in octanol]/[amount of drug dissolved in water]

where P is most commonly displayed in its logarithmic form Log P, (Christopher A. Lipinski et al., Experimental and computational approaches to estimate solubility and permeability in drug discovery and development settings; Adv. Drug Delivery Rev. (1997) 23(1-3), 3-25 (noting that most of the orally bioavailable drugs on the market seemed to have log P values less than 5)).

According to an embodiment, Ketamine may be modified so as to exhibit overall greater molecular polarity than in its unmodified form. One means for increasing the polarity of Ketamine is to link Ketamine with a water soluble magnetic carrier. Water soluble magnetic carriers were described in the context of cancer drugs in Tuszynski, J. et al., Patent Application PCT/US2004/038076. According to another embodiment, Ketamine itself may be modified to yield a much larger overall net dipole moment. Example processes are provided to convert lipophilic Ketamine to lipohobic Q-N-Ketamine by substituting the secondary amine in Ketamine to yield a quaternary ammonium salt, Q-N-Ketamine. The disclosed Q-N-Ketamine has more than simply partially positive and partially negative charges; it has full charges—a positively charged ammonium cation and negatively charged anion (a salt). Because salts cannot dissolve in nonpolar substances, the resulting Q-N-Ketamine is impermeable to the nonpolar cell walls of endothelial cells of the brain capillaries. Because the Q-N-Ketamine does not cross the BBB, it does not affect the cells of the brain. Therefore, the Q-N-Ketamine is free to work peripherally without causing undesirable psychotropic side effects.

Details about Q-N-Ketamine compositions useful in embodiments are now described. The following examples are illustrative, but not limiting, of the method and compositions of the present invention. In all examples, Ketamine is used as an initial reactant. It may be purchased commercially, e.g., Sigma-Aldrich, or can be prepared by methods known to those skilled in the art.

### Example 1

In the first example, Q-N-Ketamine is prepared by a two step process:

Step 1. Using Ketamine as a starting reagent, convert the secondary amine to a tertiary amine (Two exemplar processes for converting the secondary amine to a tertiary amine are provided for Step 1.)

Step 2. Substitution of the tertiary amine produce a quaternary ammonium salt. (Again, two exemplar syntheses are provided for Step 2.)

Each of these steps may be performed using a number of options known to those skilled in the art of medicinal and organic chemistry. Examples are provided below.

### Example 1, Step 1: Converting the Secondary Amine to a Tertiary Amine

Ketamine in its standard form contains a secondary amine Q-N-Ketamine may be prepared by first performing a reaction at the secondary amine of Ketamine to yield a tertiary amine, and then alkylating the tertiary amine to yield the quaternary ammonium salt. The reaction of the secondary amine in Ketamine may be performed for instance using a Boron-pyridine complex (BAP) mediated alkylation described in Khan, N. et al., Tetrahedron Lett., July 1996 (37) 27: 4819-4822 (the “Khan reaction”).

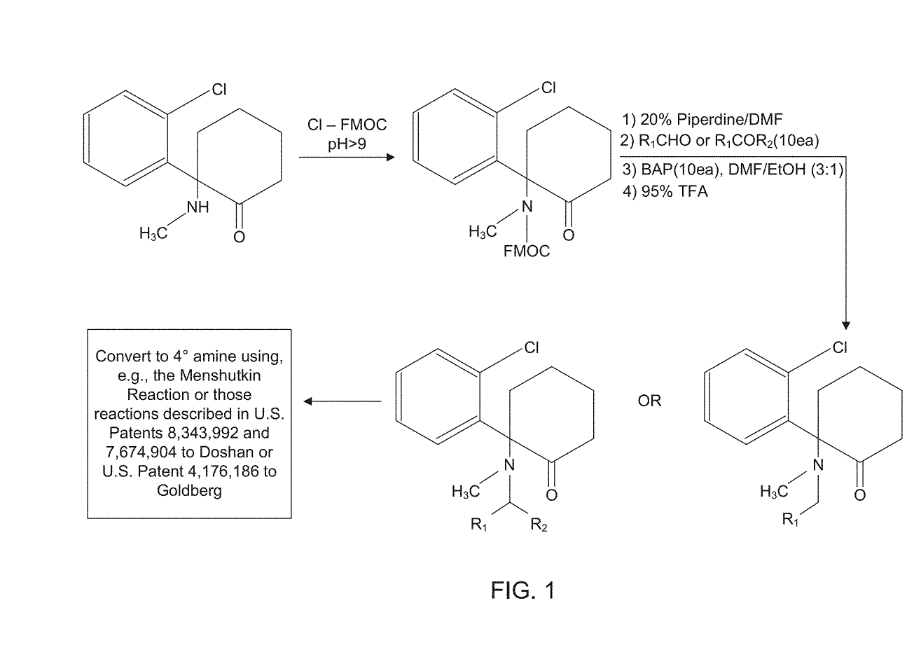

These reactions are shown in FIG. 1. The first reaction of FIG. 1 shows the addition of an amine protecting group, Fluorenylmethyloxycarbonyl chloride (“FMOC”) to Ketamine or a salt thereof, proceeding in basic conditions, preferably at a pH of greater than 9. This is followed by a BAP mediated alkylation shown in the second reaction of FIG. 1 to yield two main isomer products where R₁ and R₂ may each be Hydrogen, a C₁_{_}₆ alkyl group, or a hydrocarbon side chain as defined in Paragraph 67.

It should be noted that the conversion of Ketamine's secondary amine to a tertiary amine may be done using reactions other than via the Khan reaction. For example, the secondary amine may be converted to a tertiary amine using the Eschweiler-Clarke Reaction:

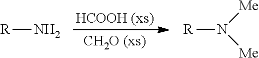

This reaction allows the preparation of tertiary methylamines from secondary amines via treatment with formaldehyde in the presence of formic acid.

The formation of quaternary amines is not possible using the Eishweiler-Clarke reaction or anticipated using the Khan reaction. For this reason, Step 2 of this Example involves a further reaction converting the tertiary amine of Step 1 to a quaternary amine, yielding a charged quaternary ammonium salt. One example for accomplishing this end is to further substitute the tertiary amine from Step 1 using the Menshutkin reaction to yield a quaternary ammonium salt.

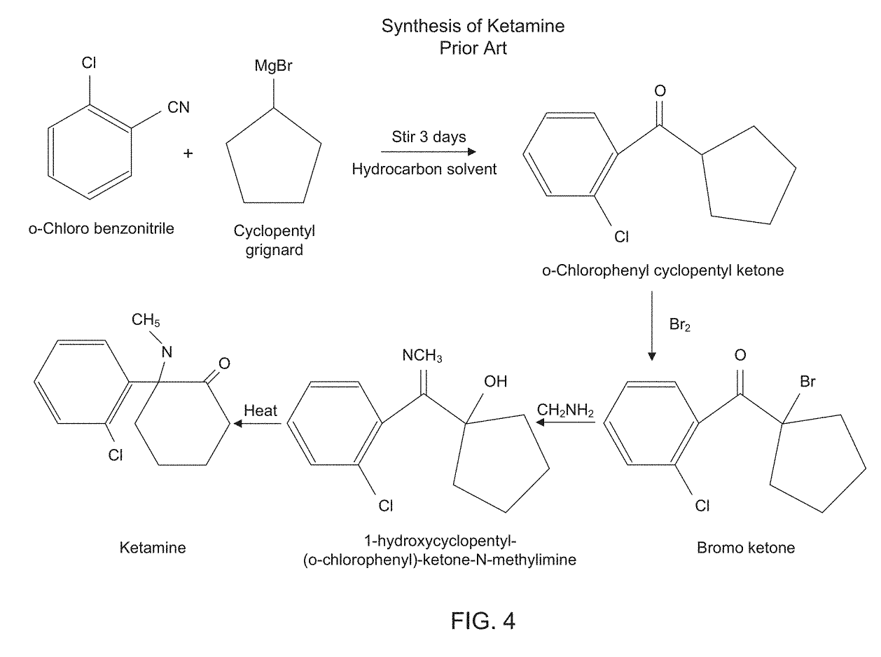

FIG. 4 depicts the Menshutkin reaction converting a tertiary amine to a quaternary ammonium salt. The reaction with the tertiary amine, for example the tertiary amine Ketamine derivative shown as a starting reagent in FIG. 4, is reacted with an alkyl halide R₄X using a suitable solvent or a solvent mixture. R₄ is preferably a C₁_{_}₆ alkyl group and X is a halogenide, such as iodide, chloride, or bromide. This yields the quaternary ammonium salt, Q-N-Ketamine.

The conversion of the tertiary amine produced in Step 1 above may also be performed using the process described, for example, in Doshan H. et al. U.S. Pat. Nos. 8,343,992 and 7,674,904. These references describe the substitution of an R group on the tertiary amine of naltrexone to yield a quaternary amine methylnaltrexone (“MNTX”), among other homologs. MNTX was first reported in the mid-70s by Goldberg et al as described in U.S. Pat. No. 4,176,186. Goldberg et al.'s U.S. Pat. No. 4,176,186, and more recently Cantrell et al.'s WO 2004/043964A2 describe a synthesis of MNTX by quaternizing a tertiary N-substituted morphinan alkaloid with a methylating agent.

The Goldberg/Doshan references use opioid tertiary amines as initial reactants and the resulting alkylated products exhibit reduced lipid solubility and resulting reduced BBB permeability. This is because the addition of the methyl group to the ring nitrogen forms a charged compound with greater polarity and less liposolubility than the original Naltrexone. This feature of MNTX prevents it from crossing the blood-brain barrier in humans. As a consequence, MNTX exerts its effects in the periphery rather than in the central nervous system. Because the resulting Q-N-Ketamine also exists as a salt, Q-N-Ketamine exhibits reduced liposolubility at the BBB.

### Example 2

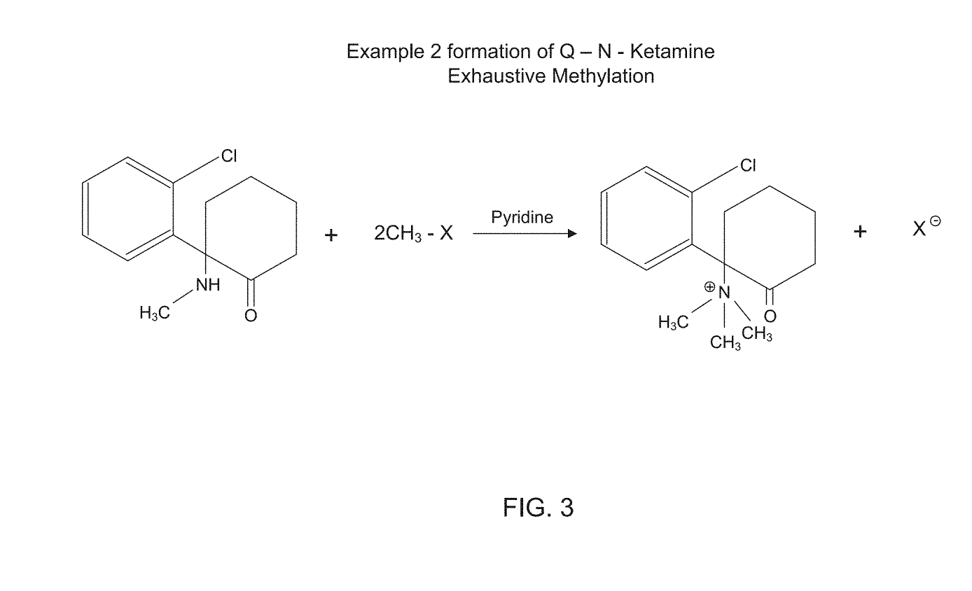

Another method for synthesizing a quaternary ammonium salt of Ketamine is via the following reaction, performed in a pyridine solvent, as shown in FIG. 3. In this reaction, X represents a halide. Applying this reaction using Ketamine as a starting reagent, the 4°-ammonium salt is prepared by repeated (exhaustive) alkylation of the amine in Ketamine.

As mentioned previously, Ketamine is highly lipid soluble and rapidly diffuses across biological membranes, including the blood-brain barrier. Natrexone, the drug modified in Doshan et al. U.S. Pat. No. 8,343,992/7,674,904 is another drug which is highly lipid soluble and readily diffuses across the blood brain barrier. A medicinal chemist skilled in the art may practice the processes described in Doshan to perform the second step of the process of Example 1. (conversion of the tertiary amine to a quaternary ammonium salt). This would open up an entirely new oral pain medication modality that would not have the central nervous system side effects. Other benefits include that the composition would be less addictive than opioids and less sedating as GABA medications are, yet with much lower side effect profiles than either.

### Example 3

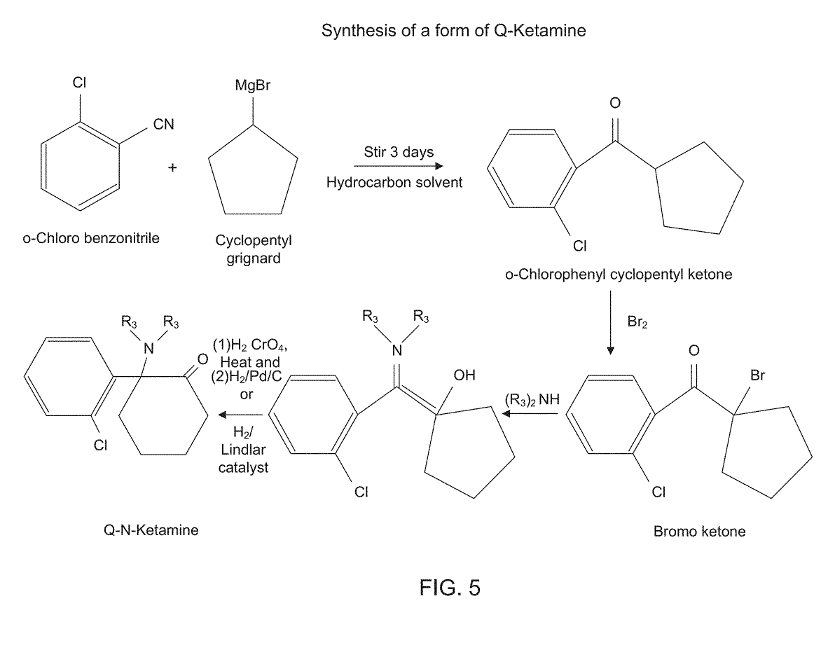

Another example for the synthesis of Q-N-Ketamine involves modifying the reactions used to prepare Ketamine so as to result in Q-N-Ketamine FIG. 4 shows one known synthesis of Ketamine FIG. 5 shows a modified synthesis of Ketamine where reaction 3 of FIG. 3 is changed to the reaction of a secondary amine with the ketone yielding an enamine. FIG. 4 also shows the oxidation of the hydroxyl group to form the ketone group of Q-N-Ketamine. This reaction uses chromic acid, although other suitable oxidizing agents may be used. Finally, the Carbon-Carbon double bond formed during the reaction of the ketone with the secondary amine may also be hydrogenated using a Platinum catalyst or Lindlar catalyst yielding the final product shown.

The compounds of the present invention may be prepared in a number of ways well known to those skilled in the art. The compounds can be synthesized, for example, by the methods described above, or variations thereon as appreciated by the skilled artisan. All processes disclosed in association with the present invention are contemplated to be practiced on any scale, including milligram, gram, multigram, kilogram, multikilogram or commercial industrial scale.

Methods of Use and Administration

One of the objects of the invention is a method for treating neuropathic pain, including peripheral neuropathy, with minimal psychotropic side effects. The term “administering” means the giving or applying of a substance, including in vivo and/or ex vivo administration. Compositions may be administered systemically either orally, buccally, parenterally, topically, by inhalation or insufflations (i.e., through the mouth or through the nose), or rectally in dosage unit formulations containing conventional pharmaceutically acceptable carriers, adjuvants, and vehicles as desired, or may be locally administered by means such as, but not limited to, injection, implantation, grafting, topical application, or parenterally. Administering can be performed, for example, once, a plurality of times, and/or over one or more extended periods.

A “patient” is a human or other animal subject that has the physiological condition. Examples of patients include, but are not limited to mammalian subjects such as humans, mice, rats, dogs, pigs, rabbits, monkeys, or apes. Examples of patients also include, but are not limited to non-mammalian subjects such as chickens, for example.

In one embodiment, these methods comprise administering to a patient Q-N-Ketamine or other quaternary derivatives of Ketamine singly or in combination with one or more other drugs. For example, Gabapentin® may be administered at or near the time of the administration of Q-N-Ketamine or other quaternary derivatives of Ketamine for providing a more patient-specific pain regimen.

Another method for treatment comprises the use of 5-10% Q-N-Ketamine, prepared in a topical formulation and administered topically at the site of the pain. Topically administered Ketamine has been used experimentally for the treatment of pain, but it is believed the circulatory system uptakes a small amount of the Ketamine, where it eventually crosses the BBB leading to some of the adverse side effects. Currently the use of topical Ketamine creams and gels between 5-10% Ketamine have had some significant success. Anecdotally, through multiple pain specialist practices, the benefits of topical ketamine is clear and has become a mainstay in treating severe peripheral neuropathies, including chemotherapy induced peripheral neuropathies, diabetic neuropathies and complex regional pain syndrome. From prior professional experience of the inventor, topical Ketamine often is more efficacious than current mainstream medications for these conditions. These topical creams however do not come without risk, adverse effects or possibility of abuse. Patient selection still has to be an important process as people have tried to ingest the topical creams to abuse Ketamine Topically, although systemic levels are very low, some people will still experience central side effects including fatigue, confusion and dizziness. The use of a topical formulation of the Q-N-Ketamine will reduce the side effects for the reasons previously stated.

The compositions disclosed herein may also be delivered via a patch. A patch is beneficial because it permits low dose infusions in specific areas that would give low systemic doses but higher concentrated doses as the site of the worst neuropathic pain areas. This would be more akin to a Lidoderm patch than a fentanyl patch, the formulation of which is known to medicinal chemists skilled in the art, as the doses would be low and peripherally acting instead of higher and systemic dosing.

Although the proper dosage of the combination products of this invention will be readily ascertainable by one skilled in the art, once armed with the present disclosure, by way of general guidance, typically a daily dosage may range from about 1 to 10 mg/kg of the Q-N-Ketamine and all combinations and subcombinations of ranges therein), per kilogram of patient body weight. Combining very low dose the Q-N-Ketamine with another medication, such as gabapentin, both of which are neuropathic pain medications, may also be an effective combination medication for neuropathic pain. Currently, most topical pain formulations combine gabapentin and ketamine for dual affects. By working to modulate both GABA and NMDA receptors, there is an increase in neuropathic pain control and improved chance in lowering noxious stimuli relayed to central pain centers.

Formulations

An example process for creating topical formulations for Ketamine. These are described in Kim, US 2013/0209585, incorporated by reference, and are appropriate for use with Q-N-Ketamine formulation and administration. The Q-N-Ketamine may also be mixed with other substances to provide a pharmaceutically acceptable dosage form. Examples of these substances include one or more excipients, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, essential oils, emollients, dissolution enhancers, dispersing agents, or combinations thereof.

The above references are incorporated by reference and are enabling for a medicinal chemist skilled in the art to use in modifying Ketamine to exhibit a sufficient degree of molecular polarity (the same as “net molecular dipole moment) so as to prohibit the Ketamine from passing the BBB and, for example, other suitable reactions and methods known in the art for reacting the secondary amine in Ketamine to form a quaternary ammonium salt may be used along with formulations suitable for delivery of the medication. The above described methods also enable a physician skilled in the art to treat neuropathic pain with the compositions disclosed while minimizing adverse side effects in patients.

Other suitable modifications and adaptations of the variety of conditions and parameters normally encountered in medicinal chemistry and which are obvious to those skilled in the art are within the spirit and scope of the invention. The compound that may be synthesized in this method of invention include, without limitation, Q-N-Ketamine or a pharmaceutically acceptable salt thereof. Therefore, there will be a counterion, which for the present application, includes the zwitterion. Preferably, the pharmaceutically acceptable salt is a halogenide, such as an iodide, a chloride or a bromide salt. “Pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine

In addition, solvents and protecting groups provided are examples. Other solvents or protecting groups known to those skilled in the art may be used in the reactions described. For example, while the amine protecting group FMOC-Cl is provided during the E, the FMOC may be introduced by other means known in the art for introducing an FMOC amine protecting group, such as FMOC-OSu, which may itself be obtained with the reaction of FMOC-Cl with the dicyclohexylammonium salt of N-hydroxysuccinimide. Alternatively, other amine protecting groups may be utilized.

Some of the compounds disclosed herein may contain one or more asymmetric centers and may thus give rise to enantiomers, diastereomers, and other stereoisomeric forms. The present invention is also meant to encompass all such possible forms, as well as their racemic and resolved forms and mixtures thereof. For example, the compositions disclosed include modifications of Ketamine using both enantiomers as initial reactants and both enantiomeric forms in the resulting Q-N-Ketamine. The chiral center of the cyclohexanone ring in Ketamine permits the existence of two enantiomers.

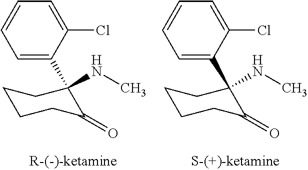

For the purpose of this disclosure, both Q-N-Ketamine and Ketamine shall encompass both configurations around the chiral center. The individual enantiomers may be separated according to methods that are well known to those of ordinary skill in the art.

As used herein, the term “alkyl” refers to an optionally substituted, saturated, straight or branched hydrocarbon having from about 1 to about 20 carbon atoms (and all combinations and subcombinations of ranges and specific numbers of carbon atoms therein), with from about 1 to about 8 carbon atoms. Alkyl groups include, but are not limited to, methyl, ethyl, n-propyl, isopropyl, n-butyl, isobutyl, sec-butyl, t-butyl, n-pentyl, isopentyl, neopentyl, n-hexyl, isohexyl, 3-methylpentyl, 2,2-dimethylbutyl, and 2,3-dimethylbutyl. The “alkylation” reaction described is preferred, but the R₁ and R₂ groups added to the secondary amine in Ketamine are not limited to saturated hydrocarbon alkyl groups.

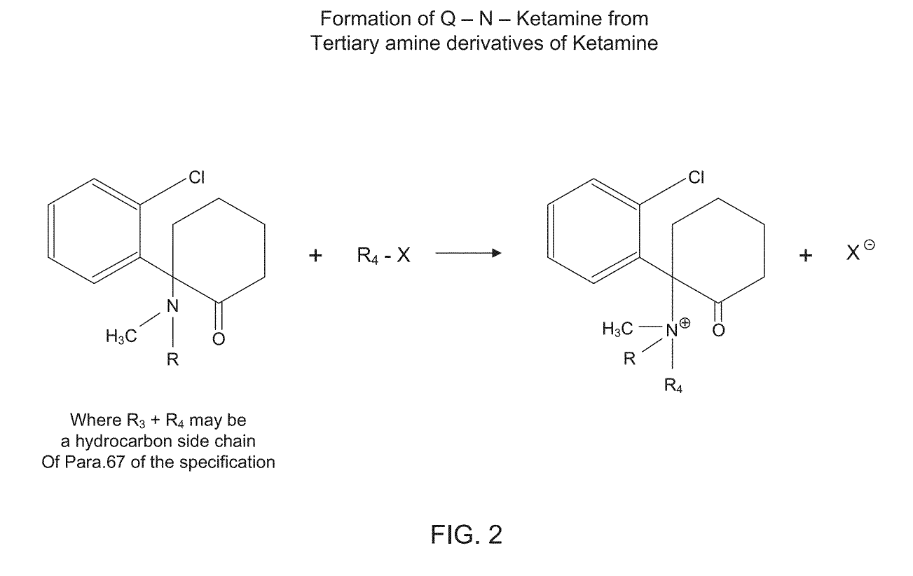

In the chemical arts, the use of the symbol “R” is widely used to designate a generic group, element, or side chain. For that reason, “R” of Para. 40 refers to generic “R” groups and are used merely to depict the Eishweiler-Clarke reaction in the general sense. In contrast, R₁ and R₂ of FIG. 1 may be either be (1) a single Hydrogen, (2) alkyl side chains, or (3) straight or branched hydrocarbon side chains having varying degrees of saturation (alkenyl and/or alkynyl portions)(referred to herein as “hydrocarbon side chain”). “R₃” (FIG. 2 and FIG. 5) and “R₄” (FIG. 2) refer to a hydrocarbon side chain. These “hydrocarbon side chains” may be substituted, such as hydrocarbon side chains having functional groups or halogens bonded to the hydrocarbon side chain.

All cited references including publications and patent documents cited in this specification are herein incorporated by reference in their entireties as if each individual publication or patent application were specifically and individually indicated to be incorporated by reference. Although the foregoing methods and compositions have been described in some detail by way of illustration and example for purposes of clarity of understanding, it will be readily apparent to those of ordinary skill in the art in light of the teachings of these methods and compositions that certain changes and modifications may be made thereto without departing from the spirit or scope of the disclosure. The present invention is not to be limited in scope by the specific embodiments disclosed in the examples, which are intended as illustrations of a few aspects of the invention, and any embodiments that are functionally equivalent are within the scope of this invention. Indeed, various modifications of the invention in addition to those shown and described herein will become apparent to those skilled in the art and are intended to fall within the scope of the disclosure.

What is claimed is:

1. A composition for treating neuropathic pain, comprising
(a) a ketamine derivative and/or homolog thereof modified so as to reduce or prevent transference across a blood-brain barrier in a patient; and a
(b) a pharmaceutically acceptable carrier.

2. The composition of claim 1, wherein the ketamine derivative and/or homolog thereof has been modified so as to exhibit overall greater net molecular dipole moment than the unmodified form of Ketamine.

3. The composition of claim 1, wherein the ketamine derivative is in a form of a pharmaceutically acceptable salt.

4. A method of reducing pain in a patient in need comprising administering a therapeutically effective amount of the composition of claim 1.

5. A ketamine derivative and/or homolog thereof modified so as to reduce transfer across a blood-brain barrier in a patient.

6. A method comprising modifying ketamine to produce a modified ketamine that comprises overall greater molecular polarity than in its unmodified form.

7. The method of claim 6, wherein modifying comprises linking ketamine with a water soluble magnetic carrier.

8. The method of claim 6, wherein modifying comprises converting the ketamine into a quaternary ammonium salt.

9. The method of claim 8, comprising performing a reaction at the secondary amine of Ketamine to yield a tertiary amine, and then alkylating the tertiary amine to yield the quaternary ammonium salt.

10. The method of claim 9, wherein the reaction of the secondary amine in ketamine comprises using a Boron-pyridine complex (BAP) mediated alkylation

11. The method of claim 10, wherein the BAP mediated alkylation comprises implementing a Khan reaction.

12. The composition of claim 1, formulated for topical administration.
